(19) United States
(12) Patent Application Publication
(10) Pub. No.: US 2022/0125327 A1
(43) Pub. Date: Apr. 28, 2022

(54) SPHYGMOMANOMETER

(71) Applicant: OMRON HEALTHCARE Co., Ltd., Muko-shi (JP)

(72) Inventors: Keita IKEDA, Kyoto (JP); Bo ZHAO, Dalian (CN); Wataru TSUNODA, Kyoto (JP); Chiaki EBISU, Kyoto (JP)

(73) Assignee: OMRON HEALTHCARE Co., Ltd., Muko-shi (JP)

(21) Appl. No.: 17/572,924

(22) Filed: Jan. 11, 2022

Related U.S. Application Data
(63) Continuation of application No. PCT/JP2020/025878, filed on Jul. 1, 2020.

(30) Foreign Application Priority Data
Jul. 19, 2019 (CN) 201910655357.6

(51) Int. Cl. A61B 5/021; H01L 41/047; A61B 5/022

(52) U.S. Cl. CPC A61B 5/02141; A61B 5/022; H01L 41/0475

(57) ABSTRACT

A sphygmomanometer includes a casing including a base and a side wall surrounding the base, a circuit board provided in the casing, a piezoelectric pump located in the casing and including a nozzle of which gas is jetted out, a first lead wire and a second lead wire each having one end electrically connected to the piezoelectric pump and an other end electrically connected to the circuit board, and a first rib and a second rib each projecting from the base toward the inside of the casing. A direction in which the first rib and second rib project is substantially parallel to a direction in which the nozzle extends. The first lead wire is press-fitted between the side wall and the first rib, and the second lead wire is press-fitted between the first rib and the second rib.

### CROSS-REFERENCE TO RELATED APPLICATION

This is a continuation application of International Application No. PCT/JP2020/025878, with an International filing date of Jul. 1, 2020, which claims priority of Chinese Patent Application No. 201910655357.6 filed on Jul. 19, 2019, the entire content of which is hereby incorporated by reference.

### TECHNICAL FIELD

The present invention relates to an electrical instrument field, and more particularly, to a sphygmomanometer.

### BACKGROUND

With the development of technology, electronic instruments have been widely used. Some of the electronic instruments include a movable element and can be driven by electrical power to move the movable element to convert electrical energy into mechanical energy. The movable element may be a vibration element, and such a vibration element can serve as a component of a pump in an electronic instrument such as an electronic sphygmomanometer. The vibration element vibrates in the pump to inject gas into an air bag of the electronic sphygmomanometer, thereby measuring blood pressure. (For example, Patent Document 1: JP 2017-115792 A)

It should be noted that the description given of the above technical background merely serves to facilitate understanding of those skilled in the art while helping to provide a clearer and more complete description of the technical idea of the present invention. It should not be recognized that the above technical idea has been known by those skilled in the art merely based on the description given in the background art of the present invention.

### SUMMARY

The inventors of the present invention pay attention to the fact that, in sphygmomanometers provided with a vibration element, noise may occur when the vibration element is activated. In such sphygmomanometers, a lead wire is connected to the vibration element to transmit an electrical signal to the vibration element. In sphygmomanometers in the related art, the lead wire is not fixed and thus largely moves in a flexible manner, the lead wire vibrates when the vibration element is activated. Accordingly, the vibrations of the lead wire itself and/or contact between adjacent members caused by the vibrations of the lead wire causes noise.

In order to solve the above-mentioned problem, the present invention, provided is a sphygmomanometer including a casing including a base and a side wall surrounding the base, a circuit board provided in the casing, a piezoelectric pump located in the casing and including a nozzle of which gas is jetted out, a first lead wire and a second lead wire each having one end electrically connected to the piezoelectric pump and an other end electrically connected to the circuit board, and a first rib and a second rib each projecting from the base toward an inside of the casing. A direction in which the first rib and second rib project is substantially parallel to a direction in which the nozzle extends, and the first lead wire is press-fitted between the side wall and the first rib, and the second lead wire is press-fitted between the first rib and the second rib.

### BRIEF DESCRIPTION OF THE DRAWINGS

The accompanying drawings are provided for facilitating further understanding of the examples of the present invention. The accompanying drawings constitute a part of the specification and are used to illustrate preferred embodiments of the present invention and explain the principle of the present invention together with the description by characters. The accompanying drawings to be described below merely illustrate some examples of the present invention, and it is obvious for those skilled in the art that other accompanying drawings can be acquired based on the accompanying drawings without any originality and ingenuity.

The present invention will become more fully understood from the detailed description given hereinbelow and the accompanying drawings which are given by way of illustration only, and thus are not limitative of the present invention, and wherein:

FIG. 1 is a top view in a casing of a sphygmomanometer according to an example of the present invention.

FIG. 2 is a partial perspective view of FIG. 1.

FIG. 3 is a schematic cross-sectional view as viewed from a direction A-A FIG. 1.

FIG. 4 is an enlarged view of a region indicated by a dotted line frame 10 in FIG. 1.

FIG. 5 is a schematic cross-sectional view as viewed from a B-B direction of FIG. 4.

FIG. 6 is a diagram schematically illustrating one variation of FIG. 5.

FIG. 7 is a diagram schematically illustrating one variation of FIG. 4.

### DETAILED DESCRIPTION

With reference to the following description and drawings, specific embodiments of the present invention will be disclosed in detail, and the application of the principle of the present invention will be illustrated. It should be understood that the embodiments of the present invention are not intended to limit the scope of the present invention. Within the spirit and scope of the appended claims, the embodiments of the present invention include many changes, modifications, and equivalents.

Namely, with reference to the drawings, the foregoing and other features of the present invention will become more apparent according to the following specification. It should also be noted that, from the specification and drawings, specific embodiments of the present invention will be disclosed in detail, and some embodiments to which the principle of the present invention is applicable will be described, but the present invention is not limited to such embodiments. Conversely, the present invention shall include all modifications, variations and equivalents falling within the scope of the appended claims.

In the examples of the present invention, the terms “first”, “second”, and the like are used to distinguish different elements by names, but do not indicate a spatial arrangement, a temporal order, or the like of the elements, and the elements are not limited by such terms. The term “and/or” includes any one and all combinations of one or more terms given in association with each other. The terms “included”, “include”, and “comprise” refer to the presence of a stated feature, element, device, or component, and do not preclude the presence or addition of one or more features, elements, devices, or components.

In the examples of the present invention, “one”, “the”, and the like indicating a single aspect include a plurality of aspects and should be understood in a broad sense as “one kind” or “one type”, but are not limited to the meaning of “one”. Further, the term “the” should be understood to include both a single aspect and a plurality of aspects, unless otherwise described above and below. Further, the term “on the basis of” should be understood as “on the basis at least in part of” unless otherwise described above or below, and the term “based on” should be understood as “based at least in part on” unless otherwise described above or below.

### First Example

Provided according to a first example of the present invention is a sphygmomanometer.

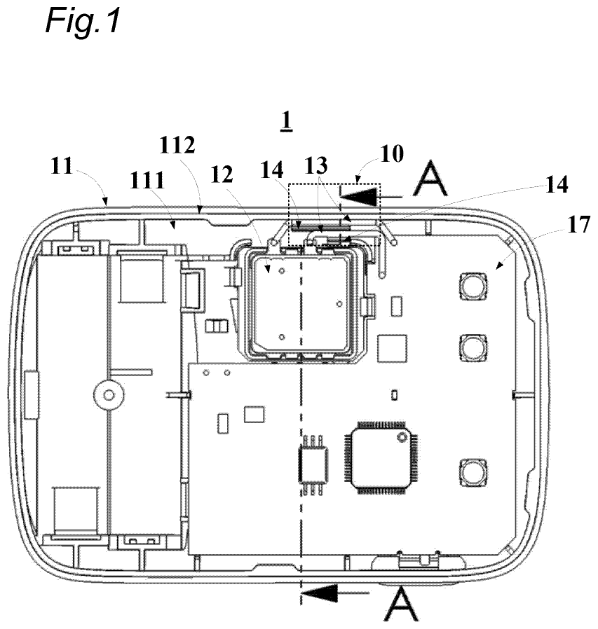

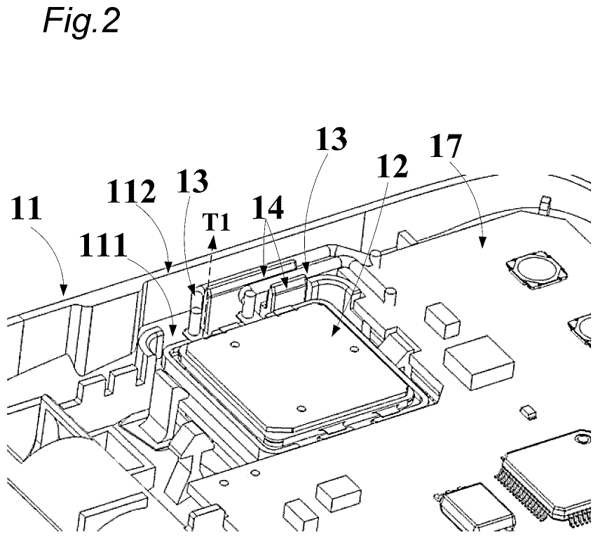

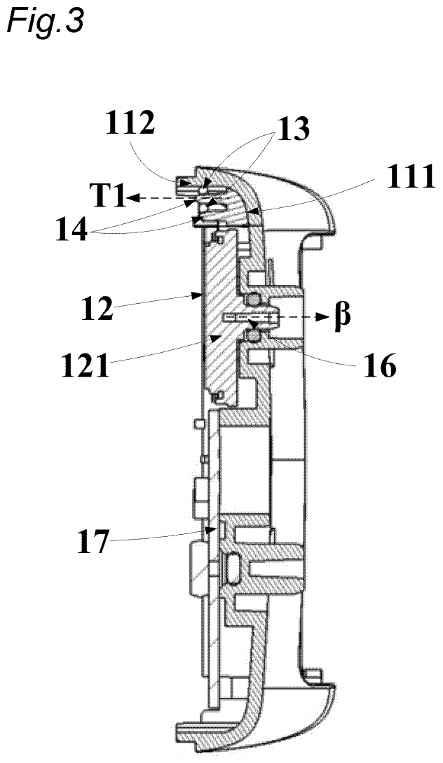

FIG. 1 is a top view of an inside of a casing of a sphygmomanometer according to an example of the present invention, FIG. 2 is a partial perspective view of FIG. 1, and FIG. 3 is a schematic cross-sectional view as viewed from an A-A direction of FIG. 1.

As illustrated in FIGS. 1, 2, and 3, a sphygmomanometer 1 includes a casing 11, a piezoelectric pump 12, a lead wire 13, a rib 14, and a circuit board 17.

According to the present example, as illustrated in FIGS. 1, 2, and 3, the casing 11 includes a base 111 and a side wall 112 surrounding the base 111, and the side wall 112 extends from a periphery of the base 111 in a direction orthogonal to the base 111. Here, the base 111 has a substantially plate shape. The plate-like base 111 may be, for example, quadrilateral in a plan view, four sides of the quadrilateral may have a predetermined curvature, and four corners of the quadrilateral may have a predetermined curvature. Further, the base 111 may have projections extending toward the inside of the casing 11 provided on an inner surface of the base 111. Such projections allow a predetermined element to be placed and/or increase strength of the base 111.

According to the present example, as illustrated in FIGS. 1, 2, and 3, the piezoelectric pump 12 is located in the casing 11. Further, the piezoelectric pump 12 may be provided on the base 111.

According to the present example, as illustrated in FIG. 3, the piezoelectric pump 12 may have a nozzle 16 of which gas is jetted out.

Further, the piezoelectric pump 12 may further include a body 121. Here, the body 121 can vibrate at a predetermined frequency, and the body 121 may include, for example, a piezoelectric element. The nozzle 16 can communicate with the outside of the casing 11. When the piezoelectric pump 12 is activated, vibrations of the body 121 cause gas to flow toward the nozzle 16, thereby allowing the gas to jet out from the nozzle 16 to the outside of the casing 11. For example, filling an air bag located outside of the casing 11 with the gas flowing through the nozzle 16 allows the sphygmomanometer 1 to measure blood pressure. A direction in which the nozzle 16 extends is a direction β as illustrated in FIG. 3, and the direction β may be substantially orthogonal to a plane on which the base 111 is located, for example.

According to the present example, as illustrated in FIGS. 1, 2, and 3, the lead wire 13 is located in the casing 11, and the lead wire 13 has one end electrically connected to the piezoelectric pump 12 and the other end electrically connected to the circuit board 17 in the casing. For example, both ends of the lead wire 13 can be fixedly connected to an electrical connection terminal (not illustrated) of the piezoelectric pump 12 and an electrical connection terminal of the circuit board by soldering (not illustrated).

According to the present example, the lead wire 13 can transmit an electrical signal between the circuit board 17 and the piezoelectric pump 12. For example, the circuit board 17 can supply power to the piezoelectric pump 12 over the lead wire 13 and transmit a control signal. Further, the circuit board 17 can further receive a signal or the like fed back by the piezoelectric pump 12 over the lead wire 13.

According to the present example, as illustrated in FIGS. 1, 2, and 3, the rib 14 projects from the inner surface of the casing 11 toward the inside of the casing 11. A storage space is provided between the rib 14 and the inner surface of the casing 11 and/or between the ribs 14. The lead wire 13 is provided in the storage space. For example, bringing the lead wire 13 into close contact with both sides of the storage space allows the lead wire 13 to be press-fitted in the storage space.

According to the present example, the rib is provided on the inner surface of the casing of the sphygmomanometer. The lead wire connected to the piezoelectric pump is provided between the ribs and/or between the rib and the inner surface of the casing. This allows the lead wire to be fixed and thus allows noise caused by vibrations of the lead wire to be greatly reduced.

According to the present example, the number of storage spaces provided for occupancy of the lead wire 13 may be one, or two or more. The storage space provided for occupancy of the lead wire 13 may be provided only between the rib 14 and the inner surface of the casing 11, or alternatively may be provided only between two adjacent ribs 14. Alternatively, the two or more storage spaces may include a storage space provided between the rib 14 and the inner surface of the casing 11, and a storage space provided between two adjacent ribs 14.

According to the present example, one storage space may be occupied by one lead wire 13, or alternatively may be occupied by two or more lead wires 13.

In the description of the present example, the sphygmomanometer 1 will be described with reference to the example illustrated in FIGS. 1 to 3.

According to the present example, as illustrated in FIGS. 1 to 3, the rib 14 may project from the base 111 toward the inside of the casing 11. The projecting direction is a direction denoted as T1 in FIGS. 2 and 3. Here, as illustrated in FIG. 3, the projecting direction T1 is substantially parallel to the extending direction β of the nozzle 16.

According to the present example, as illustrated in FIGS. 1 to 3, the storage space is provided between the rib 14 and the side wall 112 of the casing 11 and between two adjacent ribs 14. The number of the lead wires 13 is two, and lead wires 13 are each press-fitted to a corresponding one of the storage spaces. This allows the storage space to be provided by the side wall 112, thereby preventing an increase in size of the casing. Further, since each of the storage spaces is occupied by one lead wire 13, the lead wire 13 can be securely fixed.

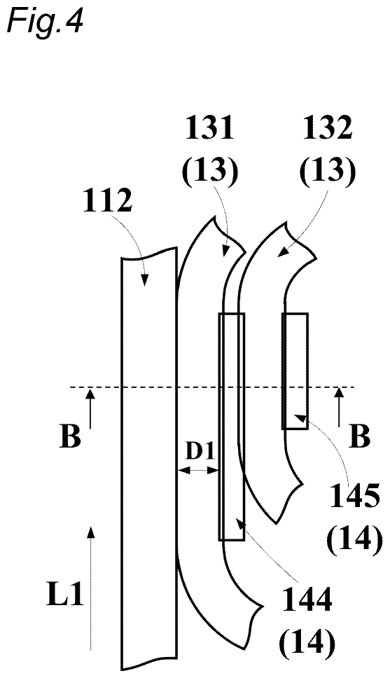

FIG. 4 is an enlarged view of a region indicated by a dotted line frame 10 in FIG. 1. As illustrated in FIG. 4, the ribs 14 may extend parallel to the side wall 112 on the base 111 (not illustrated in FIG. 4).

As illustrated in FIG. 4, the rib 14 adjacent to the side wall 112 is a first rib 144, and the rib 14 remote from the side wall 112 is a second rib 145. The lead wire 13 press-fitted between the side wall 112 and the first rib 144 is a first lead wire 131, and the lead wire 13 press-fitted between the first rib 144 and the second rib 145 is a second lead wire 132.

According to the present example, a maximum distance D 1 between the first rib 144 projecting from the base 111 and the side wall 112 may be smaller than a predetermined value, so that the rib 14 may be provided on the edge of the base 111. On the one hand, adjusting a width of the storage space between the first rib 144 and the side wall 112 allows the lead wire 13 to be securely fixed to the storage space. On the other hand, providing the first rib 144 on the edge of the base 111 can ease restrictions, due to the installation of the first rib 144, on layout positions of other elements in the casing 11. According to the present example, the predetermined value is, for example, 90% of the width of the lead wire.

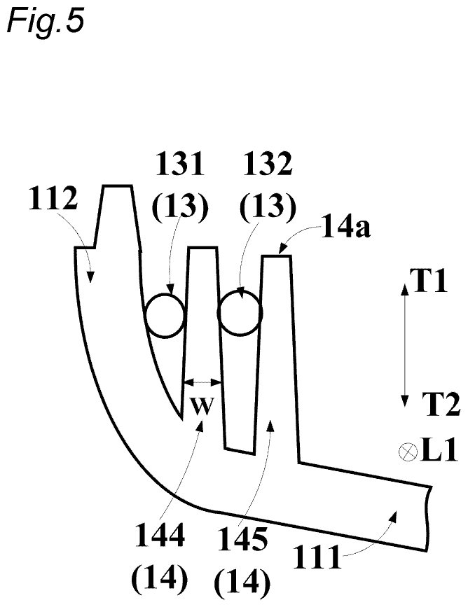

FIG. 5 is a schematic cross-sectional view taken along a line B-B in FIG. 4. As illustrated in FIG. 5, the first rib 144 and the second rib 145 have a predetermined width W. Here, the width W is a size in a direction orthogonal to both the direction T1 in which the first rib 144 and the second rib 145 project and the direction L1 in which the first rib 144 and the second rib 145 extend along the side wall 112 (as illustrated in FIGS. 4 and 5).

As illustrated in FIG. 5, the first rib 144 and the second rib 145 are structured to have the width W smaller toward a distal end 14a projecting in the direction T1 in which the first rib 144 and the second rib 145 project. This allows the lead wire 13 to be easily placed in the storage space along a direction T2 opposite to the projecting direction T1, and helps easily make the first rib 144 and the second rib 145 by molding.

Further, the present example is not limited to such a structure, and for example, the rib 14 is structured to have the width W constant in the projecting direction T1 of the rib 14. This can simplify the structure of the rib 14.

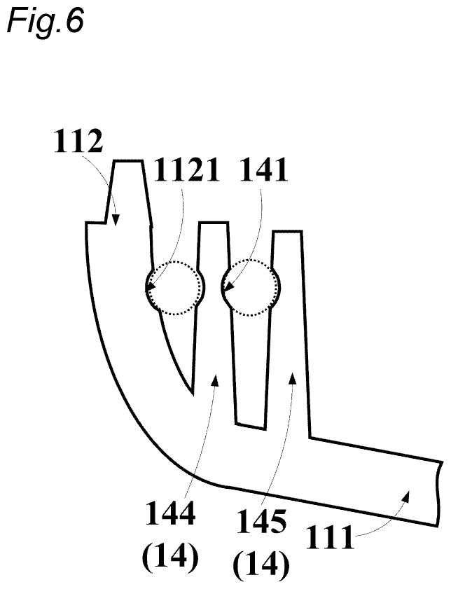

FIG. 6 is a diagram schematically illustrating another variation of FIG. 5. As illustrated in FIG. 6, a dent 141 may be provided on the side surfaces of the first rib 144 and the second rib 145. Causing the dent 141 to engage with a periphery of the lead wire 13 restricts the movement of the lead wire 13, thereby allowing an increase in anchor effect on the lead wire 13. Further, as illustrated in FIG. 6, a dent 1121 may also be provided on the side surface of the side wall 112 facing the storage space. Here, only one or both of the dents 141 and 1121 may be provided.

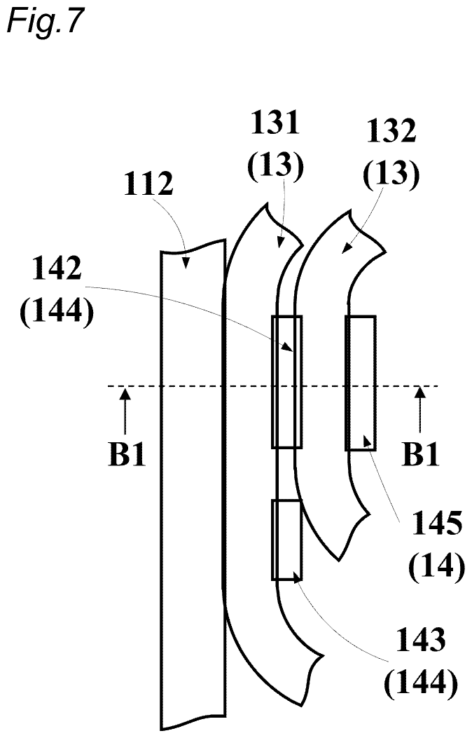

FIG. 7 is a diagram schematically illustrating one variation of FIG. 4. As illustrated in FIG. 7, at least one rib 14 may be provided at intervals in the extending direction L1 of the rib 14. For example, the first rib 144 adjacent to the side wall 112 includes a first sub rib 142 and a second sub rib 143 provided at intervals. This allows reductions in the amount of material used for the rib 14 and cost. Further, for a schematic cross-sectional view as viewed from the B1-B1 direction in FIG. 7, reference can be made to FIG. 5 or FIG. 6.

According to the present example, the sphygmomanometer 1 may further include an adhesive part (not illustrated). The adhesive part may have viscosity and may be provided at least between the first rib 144 and/or the second rib 145 and the first lead wire 131 and/or the second lead wire 132. Accordingly, the anchor effect of rib 14 on lead wire 13 can be further increased. For example, the adhesive part may be an adhesive or the like.

According to the present example, the first rib 144 and the second rib 145 may be integrally molded with the casing 11. This can simplify the manufacturing process. Further, the present example is not limited to such a structure, and may be a structure where the rib 14 and the casing 11 are fixedly connected by, for example, a connection member, and the connection member is, for example, an engagement structure, an adhesive member, or the like.

According to the present example, the connection part between the piezoelectric pump 13 and the first lead wire 131 and the second lead wire 132 may be located at an end of the piezoelectric pump 13 closest to the side wall 112. This allows the sphygmomanometer 1 to be downsized.

The sphygmomanometer 1 may further include a covering casing (not illustrated). The covering casing can cover, for example, the casing 11 and cooperate with the casing 11 to protect elements provided inside the casing 11.

According to the present example, the rib is provided on the inner surface of the casing of the sphygmomanometer, and the lead wire connected to the piezoelectric pump is provided between the ribs and/or between the rib and the inner surface of the casing. This allows the lead wire to be fixed and thus allows noise caused by vibrations of the lead wire to be greatly reduced. Further, providing the lead wire between the side wall and the rib makes it possible to avoid an increase in size of the casing. This makes it possible to reduce noise caused by the lead wire while the overall size of the sphygmomanometer remains small.

As described above, the present invention provides a sphygmomanometer in which ribs are provided on an inner surface of a casing of the sphygmomanometer, and a lead wire connected to a piezoelectric pump is provided between the ribs and between the ribs and the inner surface of the casing. This allows the lead wire to be fixed and thus allows noise caused by vibrations of the lead wire to be greatly reduced.

According to an aspect of an example of the present invention, provided is a sphygmomanometer including a casing including a base and a side wall surrounding the base, a circuit board provided in the casing, a piezoelectric pump located in the casing and including a nozzle of which gas is jetted out, a first lead wire and a second lead wire each having one end electrically connected to the piezoelectric pump and an other end electrically connected to the circuit board, and a first rib and a second rib each projecting from the base toward an inside of the casing. A direction in which the first rib and second rib project is substantially parallel to a direction in which the nozzle extends, and the first lead wire is press-fitted between the side wall and the first rib, and the second lead wire is press-fitted between the first rib and the second rib.

According to another aspect of the example of the present invention, a connection part between the piezoelectric pump and each of the first lead wire and second lead wire is located at an end of the piezoelectric pump closest to the side wall.

According to another aspect of the example of the present invention, when widths of the first rib and second rib each correspond to a size in a direction orthogonal to both the direction in which the first rib and second rib project and a direction in which the first rib and second rib extend along the side wall, the widths of the first rib and second rib are set smaller toward distal ends projecting in the direction in which the first rib and second rib project.

According to another aspect of the example of the present invention, a dent for occupancy of the first lead wire and/or the second lead wire is provided on a side surface of the first rib and/or the second rib, and/or an inner side of the casing.

According to another aspect of the example of the present invention, the sphygmomanometer further includes an adhesive part having viscosity provided at least between the first rib and/or second rib and the first lead wire and/or second lead wire.

According to another aspect of the example of the present invention, the first rib and second rib are integrally molded with the casing.

As one of the beneficial effects of the example of the present invention, the ribs are provided on the inner surface of the casing of the sphygmomanometer, and the lead wires connected to the piezoelectric pump are each provided between the ribs and/or between the ribs and the inner surface of the casing. This allows the lead wire to be fixed and thus allows noise caused by vibrations of the lead wire to be greatly reduced.

The present invention has been described above with reference to a combination of the specific embodiments. It should be understood by those skilled in the art that any of the descriptions is merely illustrative and is not construed as a limitation on the protection scope of the present invention. Those skilled in the art can make various variations and modifications to the present invention based on the principle of the present invention, but such variations and modifications also fall within the scope of the present invention. The plurality of embodiments described above can be implemented separately, but the embodiments can also be combined. Likewise, various features in different embodiments can be implemented separately, but the features in different embodiments can also be combined.

1. A sphygmomanometer comprising:
a casing including a base and a side wall surrounding the base;
a circuit board provided in the casing;
a piezoelectric pump located in the casing and including a nozzle of which gas is jetted out;
a first lead wire and a second lead wire each having one end electrically connected to the piezoelectric pump and an other end electrically connected to the circuit board; and
a first rib and a second rib each projecting from the base toward an inside of the casing,
wherein
a direction in which the first rib and second rib project is substantially parallel to a direction in which the nozzle extends, and
the first lead wire is press-fitted between the side wall and the first rib, and the second lead wire is press-fitted between the first rib and the second rib.

2. The sphygmomanometer according to claim 1, wherein a connection part between the piezoelectric pump and each of the first lead wire and second lead wire is located at an end of the piezoelectric pump closest to the side wall.

3. The sphygmomanometer according to claim 1, wherein when widths of the first rib and second rib each correspond to a size in a direction orthogonal to both the direction in which the first rib and second rib project and a direction in which the first rib and second rib extend along the side wall, the widths of the first rib and second rib are set smaller toward distal ends projecting in the direction in which the first rib and second rib project.

4. The sphygmomanometer according to claim 1, wherein a dent for occupancy of the first lead wire and/or the second lead wire is provided on a side surface of the first rib and/or the second rib, and/or an inner side of the casing.

5. The sphygmomanometer according to claim 1, further comprising an adhesive part having viscosity provided at least between the first rib and/or second rib and the first lead wire and/or second lead wire.

6. The sphygmomanometer according to claim 1, wherein the first rib and second rib are integrally molded with the casing.
